(19) United States
(12) Patent Application Publication
(10) Pub. No.: US 2013/0058903 A1
(43) Pub. Date: Mar. 7, 2013

(54) Stem-Cell Material and Method of Use

(71) Applicant: Techung Lee, Williamsville, NY (US)

(72) Inventor: Techung Lee, Williamsville, NY (US)

(21) Appl. No.: 13/514,757

(22) PCT Filed: Dec. 9, 2010
(86) PCT No.: PCT/US10/59678

§ 371 Date: Nov. 20, 2012

Related U.S. Application Data
(60) Provisional application No. 61/285,256, filed on Dec. 10, 2009.
(60) Provisional application No. 61/393,197, filed on Oct. 14, 2010.

(51) Int. Cl. A61K 35/12; C12N 5/0775; A61P 9/00; C12N 5/10

(52) U.S. Cl. 424/93.7; 435/325; 435/366; 435/372; 435/404

(57) ABSTRACT

Provided herein are mesenchymal stem cells which have been modified by the introduction of polynucleotides encoding for a mammalian Cdk1 protein. These cells do not sense in culture and are non-tumorigenic thereby providing an ongoing source of cells as well as conditioned medium. The conditioned medium from these cells can be used for tissue repair. Also provided is a method of modifying mesenchymal stem cells which can be continuously propagated in culture and are non-tumorigenic.

### CROSS-REFERENCE TO RELATED APPLICATIONS

This application claims priority to U.S. Provisional application No. 61/285,256, filed on Dec. 10, 2009, and U.S. Provisional application No. 61/393,197, filed on Oct. 14, 2010, the disclosures of which are incorporated herein by reference.

### GOVERNMENT FUNDING

This invention was made with government support under R01 HL084590 awarded by the National Institutes of Health. The government has certain rights in the invention.

### BACKGROUND OF THE INVENTION

Bone marrow mesenchymal stem cells (MSC) are being used in clinical trials for treating heart failure, myocardial ischemia, limb ischemia, immune disorders, diabetes, stroke, and several neurodegenerative diseases. Among the major clinically relevant features of MSC is their immune privileged status, allowing allogeneic or even xenogeneic applications of MSC. In spite of the tremendous potential for MSCs, three major hurdles can prevent future routine use of MSC in the clinical arena. First, MSC need to be routinely isolated from freshly collected human bone marrow or adipose tissue. Since MSCs are highly heterogeneous, MSC derived from different tissue donors or even different aspirates of the same tissue donor can vary in cell purity and therapeutic performance. These variations are highly undesirable in pharmaceutical manufacturing. Second, billions of MSC need to be produced in culture so that enough cells can be administered for clinical purpose. This in vitro amplification process causes MSC aging, which can be associated with genetic instability and reduced cell potency. Third, in vitro amplification of MSC typically requires the use of animal products such as fetal bovine serum as a source of growth factors. The use of animal products in pharmaceutical manufacturing is highly discouraged by FDA. In transitioning toward the clinical use of human MSC, additional variables such as differences in donor gender, age, and life style can potentially complicate the logistic aspect of MSC therapy. Consequently, no effective therapy exists for repairing tissue damages caused by ischemia, immune reaction, and aging using MSCs.

### SUMMARY OF THE INVENTION

In the present invention, we have modified mammalian MSCs to produce a stable stem cell line. The modified cells are obtained by introducing into the MSCs, polynucleotides encoding Cdk1 protein. These cells can be maintained and propaged in culture without the use of animal (non-human) serum or plasma. The use of a stable line of MSCs eliminates the need for repeated human tissue collection, donor-to-donor variation, and eliminates or minimizes the risk of contamination by animal pathogens. Further, the use of the modified MSCs is expected to reduce the cost of cell manufacturing.

Accordingly, in one aspect, this invention provides Cdk1 modified mammalian mesenchymal stem cells. These cells are stably reprogrammed and are non-tumorigenic. In another aspect, the present invention provides conditioned medium derived from the Cdk1 modified MSCs. The conditioned medium contains a myriad of growth factors capable of multiple biological functions, and as such is fundamentally different from traditional single-drug or single-growth factor treatment modules. The medium can be used as a therapeutic composition because the mesenchymal-stem cell derived factors can work in a cooperative fashion in tissue healing. Unlike cell-based therapy, the compositions comprising the conditioned medium or partially or fully purified fractions thereof can be conveniently administered by repeated intramuscular injection.

In another aspect, the present invention provides a method for treating or alleviating the symptoms of various pathological or physiological conditions including heart failure, myocardial ischemia, limb ischemia, immune disorders, diabetes, stroke, and neurodegenerative diseases by administrating the conditioned medium or fractions thereof of the cells of the present invention. The present technology can produce the conditioned medium cocktail cost-effectively.

### DESCRIPTION OF THE FIGURES

FIG. 1: qRT-PCR quantification of expression of human MSC growth factor and cytokine genes. RNA was isolated from unmodified (passage 4) and Cdk1-modified MSCs. cDNA was quantitatively amplified using primers corresponding to the illustrated genes. Threshold cycles are illustrated to compare levels of gene expression.

FIG. 2: Culture medium conditioned by the Cdk1-modified human MSCs promotes regeneration of cardiomyocytes in the failing hamster heart. Four-month old TO2 cardiomyopathic hamsters were injected intramuscularly with ˜3 million MSCs once (cell) or injected twice a week each with 0.5 ml of conditioned medium (conditioned for 24 hours) for four weeks (medium). Animals were sacrificed four weeks after treatments. Heart tissue sections were prepared for histological staining. Cardiomyocytes were stained with a troponin I antibody. Nuclei were stained with DAPI. Nuclear counting was performed using ImageJ software.

FIG. 3: Culture medium conditioned by the Cdk1-modified human MSCs promotes regeneration of capillaries in the failing hamster heart. Experiments were as described in FIG. 2. Cardiomyocytes were stained with a troponin I antibody. Capillaries were stained with FITC-labeled GSL-IB4. Capillary counting was performed using ImageJ software.

FIG. 4: Graph demonstrating the ability of porcine MSC-Cdk1 to undergo osteochondrogenesis, which is a hallmark of MSCs, was assessed by treating the cells with dexamethosone (Dex) and bone morphogeneic protein-7 (BMP7). Osteochondrogenic activity was determined by assays of alkaline phosphatase (ALP) activity after two weeks. The study shows that the modified pMSC retain robust potentials for osteochondrogenic differentiation as demonstrated previously for pMSC.

FIG. 5: Graph of the results from real time PCR that was performed to compare gene expression in porcine MSC-Cdk1 (pMSC-Cdk1) and passage #10 pMSC. Trophic factor genes (FGF2, FGF7, IL-6, MCP-1, TGF-b1, and VEGF) and those involved in glucose metabolism (GAPDH, GPI, HK1, LDH-A, and PFK-1) were selected. Expression of Cdk1 was also examined. β-actin gene was used as internal reference gene. The result shows that the Cdk1-engineered pMSC exhibit increased expression of trophic factor genes such as FGF7, IL-6, and VEGF. Expression of glycolytic genes such as GAPDH, GPI, and LDH-A was also enhanced.

FIG. 6: Graph comparing the therapeutic efficacy of early passage pMSC and pMSC-Cdk1. Control animals received saline injection of Hank's Buffered Saline Solution (HBSS). The study shows that the Cdk1-engineered MSC and the parental MSC are similar in their therapeutic potency as indicated by a statistically significant 20% improvement in ejection fraction one month after cell implantation in both groups.

FIG. 7: Graph demonstrating the ability of human MSC-Cdk1 (hMSC-Cdk1) to undergo osteochondrogenesis. The cells were cultured in the presence of Dex as described above. The study shows that hMSC-Cdk1 exhibited a nearly three-fold increase in ALP activity after Dex treatment for 10 days.

FIG. 8: Trophic actions of MSCs stimulate JAK/STAT3 signaling in cultured skeletal myocytes. C2C12 myocytes were plated in 6-well plates (105 cells per well). After overnight plating, cells were exposed to the indicated conditioned medium. A: Western blot assays of C2C12 cells exposed to MSC-CM for 5 min, 30 min, 6 hr, and 24 hr. B: In situ immunostaining of control medium- and MSC-CM-treated C2C12 cells (30 min incubation) using a p-STAT3 antibody (red) and β-actin antibody (green). Alexa647- and FITC-conjugated secondary antibodies were used. Nuclei were stained with DAPI. C: Western blot assays comparing the effects of porcine MSC-CM, a human embryonic kidney cell line (HEK293) CM, IL-6, IL-11, and LIF on STAT3 phosphorylation in C2C12 myocytes 30 min after treatment. Fifty ng/ml recombinant proteins were used. D: CT for human MSC IL-6-type cytokines. The CT for the house-keeping gene β2-microglobulin and GAPDH are ˜17 and ˜15, respectively.

FIG. 9: gp130/STAT3 signaling stimulates growth factor expression in cultured skeletal myocytes. C2C12 myocytes were treated with 2 and 5 mg/ml of a gp130 antibody (R&D Systems; #AF468) or control IgG for one hour at 37° C. The IgG isotype antibody was used as a negative control. Cells were then briefly rinsed and treated with MSC-CM for 30 min to assess STAT3 activation or 3 days for growth factor production. A: Western blot assays of C2C12 whole cell lysates using STAT3 antibodies, showing an inhibitory effect of the gp130 antibody on STAT3 phosphorylation. B: Analysis of C2C12 whole cell lysates using rodent-specific VEGF and HGF ELISA kits. Rat VEGF and mouse HGF were used as standards. *P<0.05 vs control medium.

FIG. 10: Activation of skeletal muscle STAT3 signaling by MSCs. MSCs were injected into the hamstring muscles of 4-month old TO2 cardiomyopathic hamsters as described. The injected muscles were harvested 3 days post-injection (n=3) for Western blotting and immunohistochemical analyses. A: Western blot assays were performed to assess STAT3 phosphorylation. Densitometric analysis revealed statistical significance (*P<0.05 vs. HBSS). B: In situ immunostaining of cryostat sections demonstrated nuclear p-STAT3 signals (pink) in the MSC-injected hamstrings. Myofibers were stained by a myosin heavy chain antibody (green). Nuclei were stained with DAPI (blue). A higher magnification (400×) of a p-STAT3-positive nucleus is also shown.

FIG. 11: Activation of the skeletal muscle growth factor network. The injected hamstrings were collected 3 days after injections, and tissue samples (n=4) were processed for qRT-PCR and ELISA assays. A: Gene expression analysis by qRT-PCR using β2-microglobulin as the reference gene. Rodent-specific PCR primers were designed to amplify the hamster sequences. *P<0.05 vs. HBSS control. B: Immunoassays of hamstring tissue homogenates for HGF, IGF-2, and VEGF using rodent-specific HGF, IGF-2, and VEGF ELISA kits. Mouse IGF-II was used as standard. Growth factor concentrations were expressed as ng per mg soluble proteins. *P<0.05 vs. HBSS control.

FIG. 12: Activation of myocardial growth factor signaling and expression. Cardiac ventricular tissues were harvested as described in FIG. 11 (n=3 per group). A: Signaling pathways were examined by Western blot assays. Statistical significance for each pathway was indicated. B: qRT-PCR assays of hamster growth factor expression 1 month post-MSC treatment. *P<0.05 compared to HBSS control. C: Assays of myocardial tissue levels of HGF and VEGF using rodent-specific ELISA kits 1 month post-MSC treatment. Growth factor concentrations were expressed as ng per mg soluble proteins. *P<0.05 vs. HBSS control.

FIG. 13: Extracardiac MSCs caused increased growth factor levels in multiple tissues. Liver (panel A), quadriceps (panel B), and brain (panel C) were harvested 1 month after the extracardiac MSC therapy (n=3). Soluble fractions of total tissue extracts were prepared and assayed by rodent-specific ELISA kits. Growth factor concentrations were expressed as ng per mg soluble proteins. *P<0.05, #P<0.001, and ̂P<0.01 compared to HBSS.

FIG. 14: WP1066 inhibits JAK/STAT3 signaling and abrogates MSC-mediated cardiac repair. A: Inhibitory effect of WP1066 on STAT3 phosphorylation in C2C12 myocytes. Cells were pretreated with WP1066 at the indicated dosages for 2 hours, following which cells were stimulated with MSC-CM for 30 min. Western blot assays were performed to determine STAT3 activation. B: The extracardiac MSC therapy was carried out as described previously (54). The four treatment groups (n=5 per group) were: WP1066, drug vehicle (PEG), MSC plus WP1066, and MSC plus drug vehicle (MSC+PEG). qRT-PCR assays of myocardial ANP and BNP expression (n=4 per group) after one month were presented. ANOVA was performed to assess statistical significance. *P<0.05 vs. PEG.

FIG. 15: WP1066 blocks MSC-mediated improvement in cardiac function. Cardiac function was determined by blind-folded echocardiography after MSC administration. A: Representative M-mode recording for the four animal groups at 4 weeks. B: LVEF at 2 and 4 weeks after MSCs. ANOVA was performed to assess statistical significance. *P<0.05 vs. PEG; †P<0.05 vs. WP1066; #P<0.05 vs. pre-injection.

FIG. 16: Inhibition of JAK/STAT3 signaling abolishes MSC-induced tissue growth factor levels. Soluble fractions of total hamstring extracts (panel A) and plasma samples (panel B) were assayed using rodent-specific HGF and VEGF ELISA kits (n=4 per group). Tissue growth factor concentrations were expressed as ng per mg soluble proteins. ANOVA was performed to assess statistical significance. *P<0.05 vs. PEG; †P<0.05 vs. WP1066; #P<0.05 vs. MSC+WP1066.

FIG. 17: Karyotype of two different hMSC-Cdk1 cells shows that it has a normal female karyotype.

FIG. 18: MSC proliferation and hypertrophy in culture. Porcine MSCs isolated from bone marrow were amplified in a medium containing 10% fetal bovine serum. Non-adherent hematopoietic and red blood cells were eliminated from the culture after trypsinization. The amplification process caused increased cell number (cell proliferation) and cell size (hypertrophy). The Cdk1 reprogramming method was observed to work with passage-0 MSC only, indicating that the signaling events causing cell hypertrophy interfere with the reprogramming function of Cdk1.

FIG. 19: RT-PCR quantification of Cdk1 expression. RNA were isolated from human MSC and hMSC-Cdk1, and expression of Cdk1 was analyzed by RT-PCR. The amplified Cdk1 transcript was indicated. The result suggests that MSC/Cdk1 was generated through gene reprogramming rather than overexpression of Cdk1.

### DESCRIPTION OF THE INVENTION

The present invention relates to stably modified mammalian MSC cells that are non-tumorigenic, and to conditioned medium that is produced by the cells. The present invention also relates to methods for use of the conditioned medium from the stably transfected MSCs in reducing or alleviating the symptoms of various diseased or physiological conditions. The terms “stably transfected”, “stably reprogrammed” and “stably modified” as used herein in conjuction with MSCs mean MSCs into which has/have been introduced polynucleotide(s) encoding for a mammalian Cdk1 protein, and which cells can be stably maintained and proliferated in culture, irrespective of whether or not the introduced polynucleotide has integrated into the genome of the cell. Thus, the cells may be genetically modified, epigeneticaly modified, or modified in some other way.

### Cells:

The cells can be from any mammalian source. In one embodiment, the cells are derived from human or porcine bone marrow. MSCs can also be obtained from other sources including adipose tissue, umbilical cord, liver, skeletal muscle and peripheral blood. The stably modified human MSCs can be maintained in culture in the presence of human serum/plasma without the need for animal plasma or serum (such as fetal bovine serum or other animal serum). The culture medium may be supplemented with recombinant human growth factors.

It is considered that the cdk encoding polynucleotides may not integrate into the genome of the MSCs. Rather, the presence of the polynucleotides enables the cells to be reprogrammed. This inference is based, in part, on the observation that in experiments in which the Cdk1 expression vector contained a green fluorescent protein marker, the marker protein was not deteced in the modified MSCs. In addition, expression of Cdk1 is not significantly changed in the reprogrammed MSCs. Thus in one embodiment, the stably modified cells are reprogrammed MSC cells and can provide a continuous source of conditioned medium. These cells can be propagated without the use of any animal product. These cells were found to be therapeutically effective in rescuing failing hamster heart upon intramuscular injections without forming tumor as determined by visual inspection and histology. In contrast, tumor formation was observed with unmodified MSCs (Am J. Physiol. 296:H1888-H1897, 2009).

The present invention also provides a method for producing stably modified MSCs. To obtain stably modified cells, early passage human bone marrow MSCs are infected with a recombinant lentivirus expressing the cell cycle regulator Cdk1 and neomycin phosphotransferase (G418 resistance marker). Infected MSCs can be efficiently selected for on a medium containing the antibiotic G418. The selected MSCs (referred to as engineered MSCs) exhibit a normal karyotype. In one embodiment, the method comprises obtaining MSCs from human marrow donors 25 years of age or younger, or porcine marrow donors 2 months of age or younger. The cells can be modified with Cdk1 (Cyclin dependent kinase 1) gene by introducing into MSCs a polynucleotide encoding Cdk1. Cdk1 is a serine/threonine kinase of approximately 34 kDa and has a highly conserved amino acid sequence. Nucleotide sequences encoding Cdk1 protein and amino acid sequences of Cdk1 protein are known in the art. It is expected that any nucleotide sequence encoding any Cdk1 protein will be suitable for use in the invention. In particular embodiments, the Cdk1 protein is a mammalian Cdk1 protein. Non-limiting examples of suitable mammalian Cdk1 proteins include human and porcine Cdk1 proteins. In one embodiment, the method comprises introducing a polynucleotide sequence encoding a human Cdk1 protein (Gene Bank accession #NM_{—}001786, (Dec. 7, 2009 entry), incorporated herein by reference) into MSCs.

In another embodiment, the polynucleotide sequence comprises the Cdk1 sequence of 894 base pairs. The Cdk1 gene can be introduced into MSCs cells using any of a variety of well known techniques and reagents. In one embodiment, the Cdk1 gene is introduced into the MSCs is present in a recombinant viral vector. In one embodiment, the viral vector is a retrovirus, one non-limiting example of which is a lentivirus. Lentiviral vectors for introducing polynucleotides into cells are well known in the art and are commercially available. The polynucleotide comprising the Cdk1 gene may or may not integrate into genome of the MSC. The polynucleotide comprising the Cdk1 gene can further comprise additional functional sequences, such as promoter elements, selectable markers, polycloning sites, and sequences for facilitating replication and/or chromosomal integration. In one embodiment, a region of the polyncucleotide encoding the Cdk1 amino acid sequence is operably linked to a promoter sequence provided as a component of the introduced polyncucleotide. Polyncucleotides comprising a Cdk1 gene for use in the invention can be constructed using standard molecular biology protocols (given the benefit of the present disclosure) and can be introduced into MSCs by, for example, standard transfection protocols.

In one embodiment, the introduced polynucleotide encoding the Cdk1 protein is a replication deficient retrovirus, such as a replication deficient lentivirus. In one embodiment, the lentivirus comprising the sequence encoding the Cdk1 protein is introduced into an MSC without also introducing a packaging vector. Packaging vectors and their use in conjunction with lentiviral vectors are well known in the art.

In one embodiment, the polynucleotide encoding a mammalian Cdk1 protein that is introduced into an MSC is provided as a component of a cell culture medium. Thus, exposure of MSC cells to a cell culture medium that comprises the polynucleotide encoding the mammalian Cdk1 protein can be used for MSC transfection. In one embodiment, the polynucleotide encoding the mammalian Cdk1 protein is present in replication deficient lentiviral particles in the cell culture medium that is used to transfect the MSC cells.

It is preferable to use early passage or unpassaged MSC cells for introduction of polynucleotides encoding Cdk1 protein. In one embodiment, the MSC cells are used without passaging (termed as passage 0). In another embodiment, passage 1 MSC cells are used. Passage 1 cells generally means that fresh cells are grown in culture, trypsinized and replated for propagation. Similarly, passage 2, 3, 4 and so on can be obtained.

Thus, in one embodiment, the present invention provides a method for generating modified mesenchymal stem cells. The method comprises the steps of a) obtaining mesenchymal stem cells from bone marrow, removing blood cells from the sample, without passaging the cells, obtaining non-adherant MSCs, and d) introducing into said cells a composition comprising polynucleotide encoding for a mammalian Cdk1 protein.

Generally, MSCs exhibit gradually reduced cell growth and eventually become senescent after several passages in culture, necessitating repeated human tissue collections. However, the cells of the present invention, retain their youthful features. The modified porcine and human MSCs have been propagated in culture for at least 50 and 20 passages, respectively, without losing their ability to grow. The use of the MSCs thus eliminates the need for repeated human tissue collection. Further, the use of the MSCs is expected to reduce the cost of cell manufacturing.

### Cell Culture

The present invention also provides conditioned medium as well as compositions comprising conditioned medium from stably modified mammalian MSC cells. The Cdk1 transfected cells can be cultured in monolayer, beads (i.e., two-dimensions) or in three-dimensional culture systems.

The cell culture medium in which the ckd-1 transfected cells are cultured can be any standard cell culture medium which provides adequate nutrition to the Cdk1 transfected cells. Before being conditioned (such as before being added to the cells), the medium is termed as “pre-conditioned medium”. Suitable cell media include, but are not limited to Dulbecco's Modified Eagle's Medium (DMEM), Ham's F12, RPMI 1640, Iscove's, McCoy's and other media formulations readily apparent to those skilled in the art. Such media can be easily prepared or obtained from commercial sources. Details of cell culture media and methods can be found in Methods For Preparation of Media, Supplements and Substrate For Serum-Free Animal Cell Culture Alan R. Liss, New York (1984) and Cell &Tissue Culture: Laboratory Procedures, John Wiley & Sons Ltd., Chichester, England 1996. The medium may be supplemented, with components, such as vitamins, growth factors, proteins, sugars, anti-oxidants, etc. as necessary to support the desired cell culture. Additionally serum, such as bovine serum, which is a complex solution of albumins, globulins and growth factors may be added if desired. However, for human use, it is preferred that animal products such as animal serum be avoided. Instead, human plasma can be added in the same amount as animal serum. Hormones, growth factors or other agents may be added into the medium.

### Conditioned Media

The cells can be cultured by standard methods using aseptic processing and handling. The cells are cultured in the desired culture medium. The conditioned media obtained from the cells should be processed under sterile conditions or sterilized as needed. When appropriate (i.e., once the medium is conditioned so that extracellular proteins such as growth factors have reached desirable levels in the medium) the “conditioned” medium can be collected. In one embodiment, the conditioned medium is collected after 3, 6, 12, 18, 24, 30, 36, 42, 48, and all hours therebetween. In one embodiment, it is collected anytime between 2 hours and 60 hours of incubation.

The MSC-derived cocktail is unique in that it contains a myriad of trophic factors capable of multiple biological functions, and as such is fundamentally different from traditional single-drug or single-growth factor treatment modules. Synergistic therapeutic effects can be obtained with the cocktail since the multiple-stem cell derived trophic factors can work in a cooperative fashion in tissue healing. The beneficial effects contributed by these trophic factors may include, but are not limited to, promotion of cell survival and cell differentiation, angiogenesis, anti-inflammation, and anti-fibrosis. Unlike cell-based therapy, the cocktail can be administered by repeated intramuscular injection conveniently.

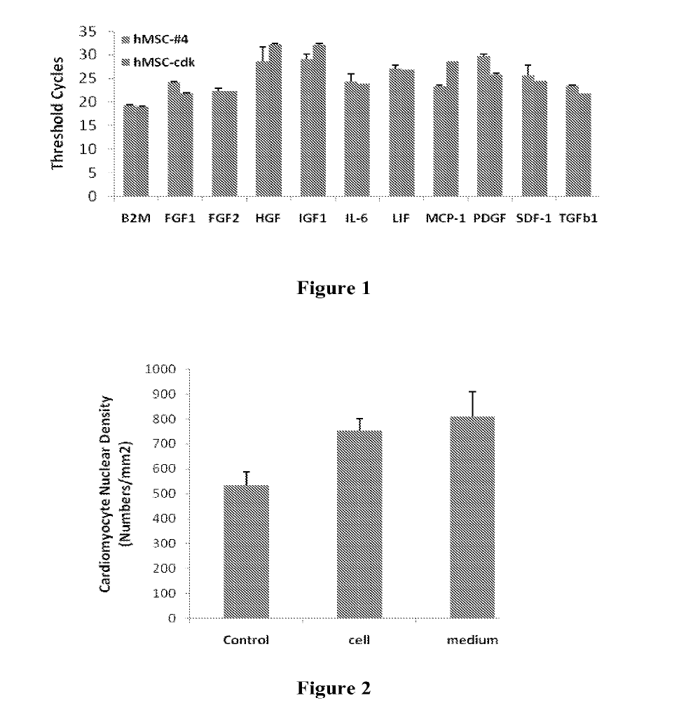

Growth factors or biomolecules reported to be produced by unmodified MSCs in culture include Angiopoietin-1; BDNF (brain-derived neurotrophic factor); BMP-7 (bone morphogcncic factor-7); CCL2 (MCP-1 or monocyte chemotactic protein-1); EGF (epidermal growth factor); ENA-78 (epithelial cell-derived neutrophil-activating peptide-78); Erythropoietin; Factor H; FGF-1, -2, -4, -9 (fibroblast growth factor); GCP-2; G-CSF (granulocyte-colony stimulating factor), M-CSF (macrophage-colony stimulating factor), GM-CSF (granulocyte/macrophage-colony stimulating factor); GDF-8 (growth and differentiation factor-8), GDF-9 (growth and differentiation factor-9); GRO; HGF (hepatocyte growth factor); Indoleamine 2,3-dioxygenase; IGF-1 (insulin-like growth factor-1), IGF-II (insulin-like growth factor-2); IGFBP-1 (IGF-binding protein), -2, -3, -4; IL-1β (interleukin-1β); IL-6 (interleukin-6); IL-8 (interleukin-8); IL-10 (interleukin-10); IL-11 (interleukin-11); CXCL10 (IP-10); KGF (FGF-7); LIF (leukemia inhibitory factor); MIF-1 (macrophage migration inhibitory factor-1); MIP-3a (macrophage inflammatory protein-3a); MMP-1 (matrix metalloproteinase), -2, -3, -9; MT1-MMP (membrane type-matrix metalloproteinase), MT3-MMP; OSM (oncostatin-M); Osteoprotegerin (OCIF); NGF (nerve growth factor); PARC (MIP-4); PGE2 (prostaglandin E2); PlGF (placental growth factor); SCF (stem cell factor); SDF-1 (stromal-derived factor-1); Sfrp2 (secreted frizzle-related protein-2); Stanniocalcin-1; TGFβ-1, -2, -3 (transforming growth factor); TIMP-1, -2 (tissue inhibitor of metalloproteinase); TNF-β (tumor necrosis factor); TSG-6 (tumor necrosis factor-stimulated gene sequence-6); VEGF (vascular endothelial growth factor). We have already determined (by PCR) that the genetically modified cells of the present invention produce EGF-1, EGF-2, HGF, IGF-1, IL-6, LIF, MCP-1, PDGF, SDF-1, and TGF β1. (FIG. 1). Additionally, we have also identified the presence of SDF-1, IL-11, and VEGF in the genetically modified MSC conditioned medium by ELISA assays. Therefore in one embodiment, this invention provides a neat (unprocessed) conditioned medium from the cells provided herein or partially or fully purified fractions of the medium comprising one or more of the following factors: Angiopoietin-1; BDNF; BMP-7; CCL2 (MCP-1); EGF; ENA-78; Erythropoietin; Factor H; FGF-1, -2, -4, -9; GCP-2; G-CSF, M-CSF, GM-CSF; GDF-8, GDF-9; GRO; HGF; Indoleamine 2,3-dioxygenase; IGF-1, IGF-11; IGFBP-1, -2, -3, -4; IL-1β; IL-6; IL-8; IL-10; IL-11; CXCL10 (IP-10); KGF (FGF-7); LIF; MIF-1; MIP-3a; MMP-1, -2, -3, -9; MT1-MMP, MT3-MMP; OSM; Osteoprotegerin (OCIF); NGF; PARC (MIP-4); PGE2; PlGF; SCF; SDF-1; Sfrp2; Stanniocalcin-1; TGF β-1, -2, -3; TIMP-1, -2; TNF-β; TSG-6; VEGF.

If desired, the conditioned medium can be processed to concentrate selected components. For example, the medium may be concentrated 10 to 20 fold using a positive pressure concentration device (such as a device having a filter with a 0.2 or 0.45 μm cut-off (Amicon, Beverly, Mass.). Also, the conditioned medium may be further processed for product isolation and purification to remove unwanted proteases, for example or purification and isolation of useful growth factors and the like. Methods of purification include gel chromatography, ion exchange, affinity chromatography HPLC purification and the like.

In one embodiment, the conditioned medium from the stably modified MSCs can be collected once the cells are over 70% confluent. In various embodiments, conditioned medium can be collected when the cells are between 70 to 100% (and all integers therebetween) confluent. In one embodiment, the cells are 80-90% confluent. Once collected, the conditioned medium is filtered to remove debris. As discussed above, the filtered medium can be used neat or can be processed to remove and/or concentrate desired components. The conditioned medium with or without processing can be used fresh or can be stored (at refrigerator or freezer temperatures) for later use.

Accordingly, in one aspect of the invention, the invention provides conditioned medium that has previously supported the growth of Cdk1 stably modified MSCs. In one embodiment, the conditioned medium is one that has previously supported the growth of human MSCs.

### Uses of Conditioned Medium

In one embodiment, the present invention provides a pharmaceutical composition for administration to an individual in need of such administration. Pharmaceutical carriers are well known in the art and can include glycerol, glycogen, maltose and the like or organic polymer base materials, such as polyethylene glycol and hyaluronic acid as well as non-fibrillar collagen, preferably succinylated collagen and the like. The pharmaceutical composition comprises conditioned medium free of MSCs or any other type of cells. The conditioned medium has previously supported the growth of genetically modified MSCs of the present invention. In one embodiment, the conditioned medium has previously supported the growth of Cdk1 modified human MSCs.

In one embodiment, the conditioned medium is produced by incubating the stably modified cells of the present invention with cell culture medium which is free of animal products like fetal bovine serum, but contains human serum/plasma obtained from the intended recipient of the therapy.

The conditioned media of the invention can be formulated into injectable, oral or topical preparations. Alternatively, products derived from the conditioned media can be formulated into injectable, oral or topical preparations. Additionally, biologically active substances, such as proteins, drugs or any other agents, can be incorporated in the compositions of the present invention for release or controlled release of these active substances after administration of the composition.

The pharmaceutical formulations of the invention may be delivered to a patient via a variety of routes using standard procedures well known to those of skill in the art. For example, such delivery may be site-specific, oral, nasal, intravenous, subcutaneous, intradermal, transdermal, intramuscular or intraperitoneal administration. Also, the compositions may be formulated to function as controlled, slow release vehicles. In one embodiment, the composition can be administered twice a week or more or less frequently until a desired effect is achieved.

MSC conditioned medium therapy can be used alone or in conjunction with existing therapeutic protocols for the treatments of heart failure, myocardial ischemia, limb ischemia, immune disorders, diabetes, stroke, and many neurodegenerative diseases. In addition, therapeutic devices or implants such as stents can be coated with the conditioned medium to maximize their therapeutic benefits.

The following examples are provided to illustrate the invention and are not to be considered as limiting in any way.

### Example 1

We conducted a series of genetic engineering trials using porcine bone marrow MSCs. We also tested human bone marrow MSCs. Commercially available lentiviral vector DNA (#EX-B0070-Lv21) and a packaging vector DNA pLV-PK-01 were purchased from GENECOPOETA (Germantown, Md.). The DNA was transiently transfected into HEK293 cells using a standard calcium-phosphate method. Culture medium was harvested after one week and used to transfect MSCs. MSCs were incubated with the HEK293 culture medium for 3 hours, following which cells were washed twice with Hank's Balanced Salt Solution and refed with a regular growth medium.

Porcine and human MSCs were maintained in DMEM/F12 supplemented with 10% fetal bovine serum (FBS) and Mesenpro RS medium, respectively. Detailed MSC culture conditions have been documented. The following experiments were performed.

### Experiment 1

Porcine MSCs (passage 6) were plated on 35-mm dishes and grown to ˜80% confluency. Cells were then transfected with ˜5 μg of plasmid DNA vectors expressing Bcl-2, Ras-R12, VEGF, or YAF2 cDNA using a commercial DNA transfection kit (Biomedical Research Service, Buffalo, USA). These genes were selected for engineering based on their pro-survival and/or growth-regulating properties. Transfected MSCs were selected with 0.1-0.5 mg/ml G418. No viable cells were obtained from this trial.

### Experiment 2

Porcine MSCs (passage 5) were transfected with plasmid DNA vectors expressing Bcl-2 or Ras-R12 cDNA with an additional glycerol shock step. Transfected MSCs were selected with 0.2 mg/ml G418. No viable cells were obtained from this trial.

### Experiment 3

Porcine MSCs (passage 8) were transfected with plasmid DNA vectors expressing Bcl-2, Ras-N17, VEGF, Raf, or anti-sense SRF cDNA with an extended transfection time. Transfected MSCs were selected with 0.2 mg/ml G418. No viable cells were obtained from this trial.

### Experiment 4

Porcine MSCs (passage 5) were transfected with plasmid DNA vectors expressing Bcl-2 or SRF-DM5 cDNA. Transfected MSCs were selected with 0.4 mg/ml G418. No viable cells were obtained from this trial.

### Experiment 5

Porcine MSCs (passage 7) were transfected with the pSFFV-Bcl-2 expression vector overnight. Transfected MSCs were selected with 0.2 mg/ml G418. No viable cells were obtained from this trial.

### Experiment 6

Human MSCs (passage 2) from donor CF17 were transfected with plasmid DNA vectors expressing Bcl-2 or Tert cDNA, and cells were selected with G418 and puromycin, respectively. No viable cells were obtained from this trial.

### Experiment 7

Human MSCs (passage 4) from donor CF17 were transfected with plasmid DNA vectors expressing VEGF, Ras-R12, PKC, PKA, NF-kB p65, IkB, Oct4P1, or anti-sense SRF cDNA, and cells were selected with G418 after transfection. No viable cells were obtained from this trial.

### Experiment 8

Porcine MSCs (passage 0) were transfected with plasmid DNA vectors expressing Bcl-2 or Tert cDNA. Transfected MSCs were selected with G418 or puromycin. No viable cells were obtained from this trial.

### Experiment 9

Porcine MSCs (passage 4) were infected with a lentiviral vector expressing EGFP overnight to optimize gene expression efficiency and time course.

### Experiment 10

Porcine MSCs (passage 1) were infected with lentiviral vectors expressing Bcl-2 or cyclin A. Cells were selected with G418. No viable cells were obtained from this trial.

### Experiment 11

Porcine MSCs were infected with lentiviral vectors expressing EGFP, Bcl-2, Cdk1, or cyclin A. Cells were selected with 0.1 mg/ml G418 for three days followed by 0.2 mg/ml G418 for three days and 0.1 mg/ml G418 for 6 days. Several colonies were found to emerge from the Cdk1 group. No viable cells were observed with the EGFP, Bcl-2, and cyclin A groups.

### Experiment 12

The experiment performed in Experiment 11 was repeated. Several colonies were again found to emerge from the Cdk1 group. These engineered porcine MSCs were expanded and aliquots of the cells were frozen for further characterizaton.

### Experiment 13

Human MSCs from donor CM25 were infected with the Cdk1 lentiviral vector, and cells were selected with 0.15 mg/ml G418 after infection (i.e., transfection). However, this G418 dosage was found to be too toxic to the cells.

### Experiment 14

Human MSCs (passage 4) from donor AF20 were infected with lentiviral vectors expressing Bcl-2, cyclin A, or Cdk1, and cells were selected with 0.2 mg/ml G418 after infection. No viable cells were obtained from this trial.

### Experiment 15

Human MSCs (passage 1) from donor AF22 were infected with the Cdk1 lentiviral vector, and cells were selected with 0.05 mg/ml G418 after infection. Several colonies emerged after G418 selection. These engineered human MSCs were expanded and aliquots of the cells were frozen for further use.

### Experiment 16

Repeat the experiment performed in Experiment 15 using human MSCs (passage 0) from AF22 donor again. G418 selection was not used due to the observed toxicity. Several fast growing and expanding colonies emerged after 10 days. These engineered human MSCs were expanded and aliquots of the cells were frozen for further use.

Characterization of Cdk1-Engineered Porcine and Human MSC (pMSC-Cdk1 and hMSC-Cdk1)

### Experiment 17

The ability of pMSC-Cdk1 to undergo osteochondrogenesis, which is a hallmark of MSCs, was assessed by treating the cells with dexamethosone (Dex) and bone morphogeneic protein-7 (BMP7). Osteochondrogenic activity was determined by assays of alkaline phosphatase (ALP) activity after two weeks. The study shows that the engineered MSCs retain robust potentials for osteochondrogenic differentiation as demonstrated previously for pMSCs.

### Experiment 18

Real time PCR was performed to compare gene expression in pMSC-Cdk1 and passage #10 pMSC. Trophic factor genes (FGF2, FGF7, IL-6, MCP-1, TGF-b1, and VEGF) and those involved in glucose metabolism (GAPDH, GPI, HK1, LDH-A, and PFK-1) were selected. Expression of Cdk1 was also examined. β-actin gene was used as internal reference gene. The result shows that the Cdk1-engineered pMSC exhibit increased expression of trophic factor genes such as FGF7, IL-6, and VEGF. Expression of glycolytic genes such as GAPDH, GPI, and LDH-A was also enhanced. Expression of Cdk1 was increased by ˜4 fold in pMSC-Cdk1.

### Experiment 19

We compared the therapeutic efficacy of early passage pMSC and pMSC-Cdk1. Control animals received saline injection (HBSS). The study shows that the Cdk1-engineered MSC and the parental MSC are similar in their therapeutic potency as indicated by a statistically significant 20% improvement in ejection fraction one month after cell implantation in both groups.

### Experiment 20

The ability of hMSC-Cdk1 to undergo osteochondrogenesis was assessed by culturing the cells in the presence of Dex as described above. The study shows that hMSC-Cdk1 exhibited a nearly three-fold increase in ALP activity after Dex treatment for 10 days.

### Example 2

We tested the ability of hMSC-Cdk1 to grow in culture without the use of FBS. The base medium is DMEM/F12 as used for pMSCs. The medium was supplemented with 15% human cord blood serum, human cord blood plasma, adult human serum, or adult human plasma in the presence of various recombinat human growth factors. The effect of heat inactivation of serum/plasma (56° C. 30 min) was also investigated. We found that heat inactivation generally enhanced the performance of cord blood plasma and adult human plasma. A medium supplemented with 15% human serum/plasma and 50 ng/ml FGF2 and EGF supports the expansion of the modified human MSCs. Addition of growth factors such as PDGF and TGFβ may further enhance the growth potential. Given that human serum/plasma from different donors may vary in their performance, as observed with FBS, commercially available serum-free MSC medium may also be used. A summary of some advantages of using MSC-Cdk1 for clinical application is provided in the Table, which is based on a comparison of MSC and MSC-Cdk1 cells.

TABLE

| | | Animal | | | |
|---|---|---|---|---|---|
| | Tissue | Product | Cellular | Consistent | Cost |
| | Collection | Dependence | aging | Performance | effective |
| MSC | Yes | Yes | Yes | No | No |
| MSC- | No | No | No | Yes | Yes |
| Cdk1 | | | | | |

### Example 3

Cardiac therapeutic effects are assessed by transthoracic echocardiography and histological analysis of hamsters using techniques as outlined in the Description of the Figures. For transthoracic echocardiography, animals were anesthetized by intraperitoneal injection of xylazine (2 mg/kg) and ketamine (25 mg/kg), and remained semi-conscious during the measurement procedure. Multiple M-mode images were obtained from the short axis view of the left ventricle at the level of the papillary muscles with a GE Vingmed echo machine using a 10-MHz transducer. From this image, left ventricular end-systolic dimension (LVDs) and left ventricular end-diastolic dimension (LVDd) were measured. These dimensions were measured and averaged from at least two consecutive cardiac cycles. For histological analysis, ventricular cross sections 5 μm thick were obtained using a cryostat, fixed in acetone:ethanol mixture (3:1 ratio) for 5 minutes. Sections were blocked with Serum Free Protein Block (Dako) for 30 minutes. FITC-labeled GSL-IB4 lectin diluted 1:100 was incubated with the tissue sections overnight at 4° C. Cardiomyocytes were stained with a rabbit TnI antibody (#sc15368; Santa Cruz) the next day for 3 hours. Sections were then incubated with a Texas Red conjugated anti-rabbit secondary antibody for 1 hour and then mounted using Vectashield's Mounting Medium with DAPI (Vector Laboratories). Images were taken in 15-25 random fields using Zeiss's Axioimager fluorescence microscope at 200× magnification. The number of capillaries and total nuclei count (DAPI channel) were quantified by ImageJ software using the analyze particle feature. Non-cardiomyocte nuclei quantified from the merged images by their lack of TnI staining were subtracted from total nuclei count to determine cardiomyocyte nuclear density. Black areas from images were subtracted using Photoshop aided quantification of black pixels to calculate total tissue area. Capillary and cardiomyocyte nuclear density were normalized to total tissue area in mm².

We show that MSC-conditioned medium upon repeated intramuscular injections possesses therapeutic effects for cardiac repair. We also indicate that the IL-6-type cytokines are required for the therapy. Expression of key growth factor and cytokine genes by unmodified and Cdk1-engineered hMSCs were compared by qRT-PCR (FIG. 1). Threshold cycle analysis showed that a passage-4 hMSCs and Cdk1 hMSCs are similar in the expression of the trophic factors. In particular, expression of IL-6 and LIF, two members of the IL-6-type family, are nearly identical, suggesting that the engineered hMSC line is therapeutically potent.

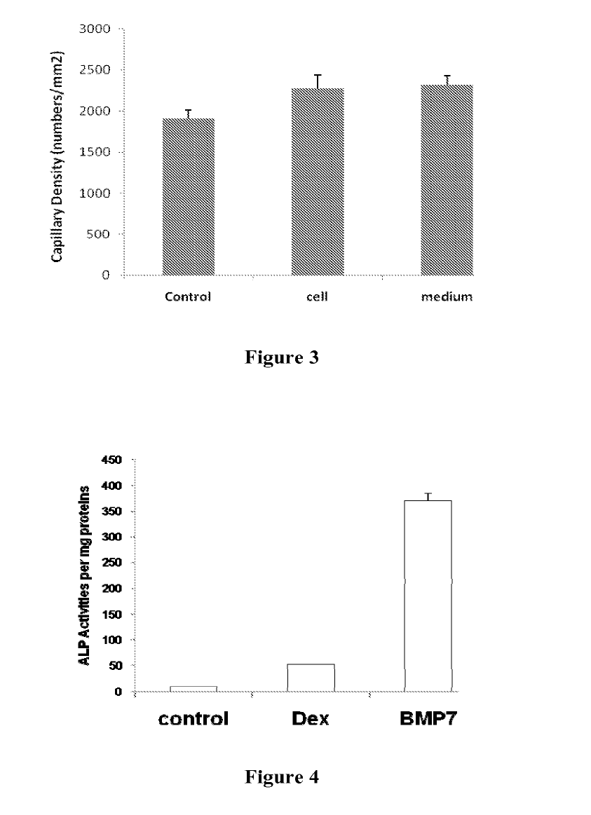

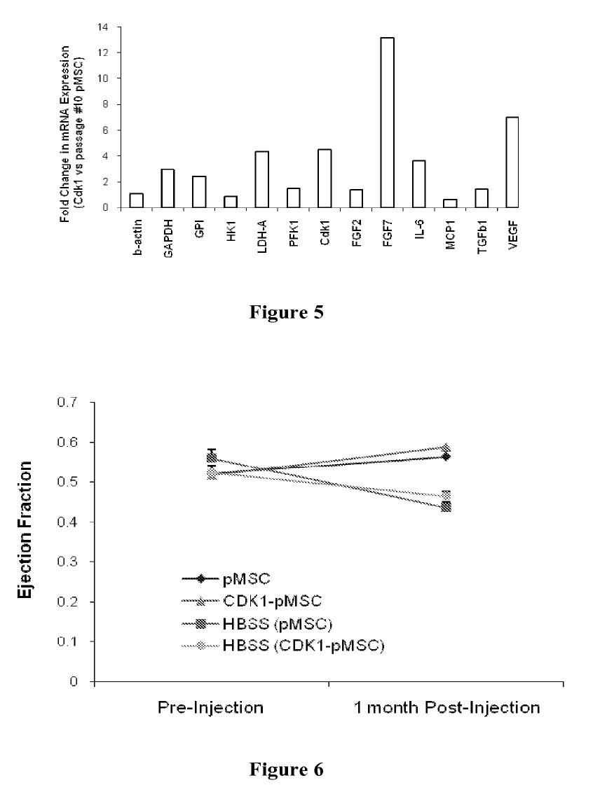

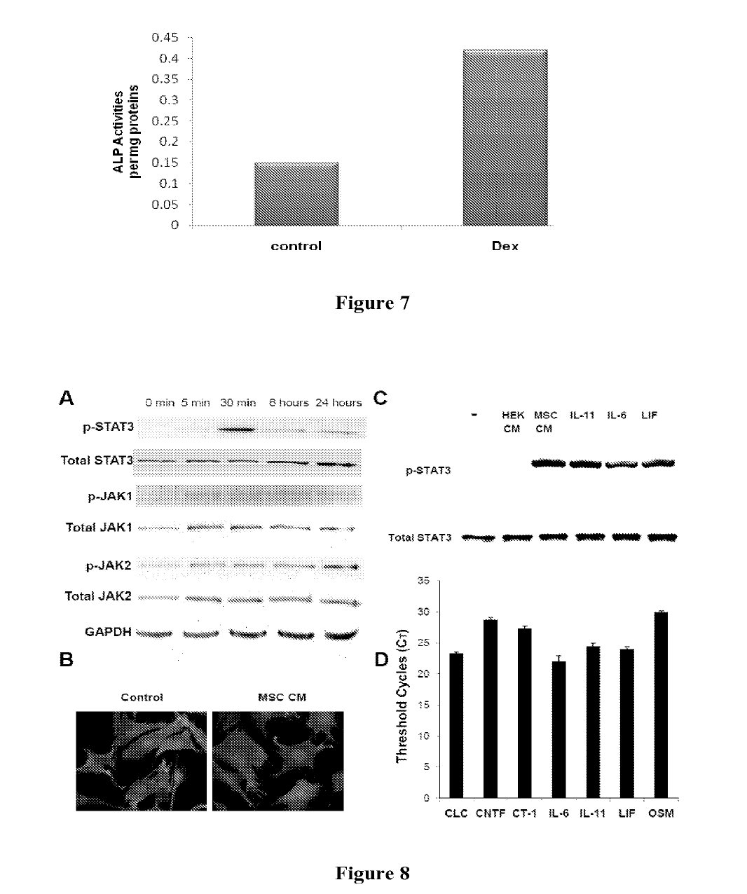

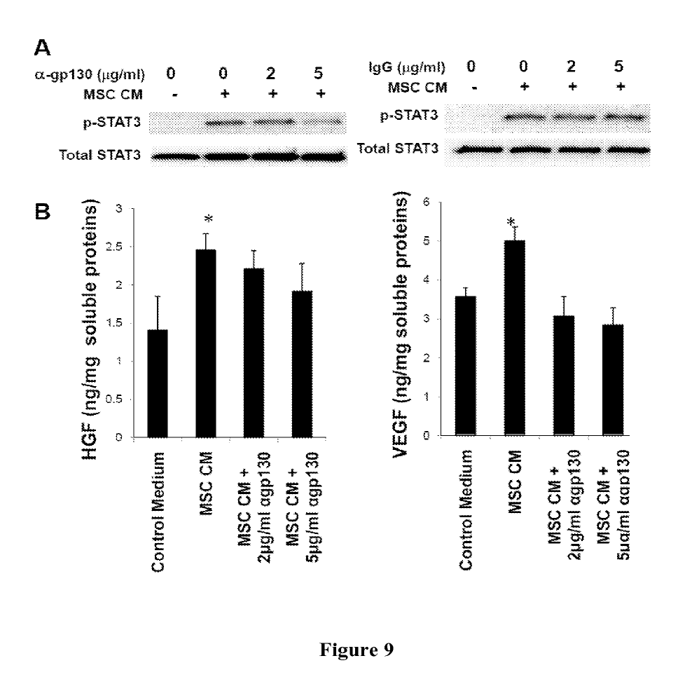

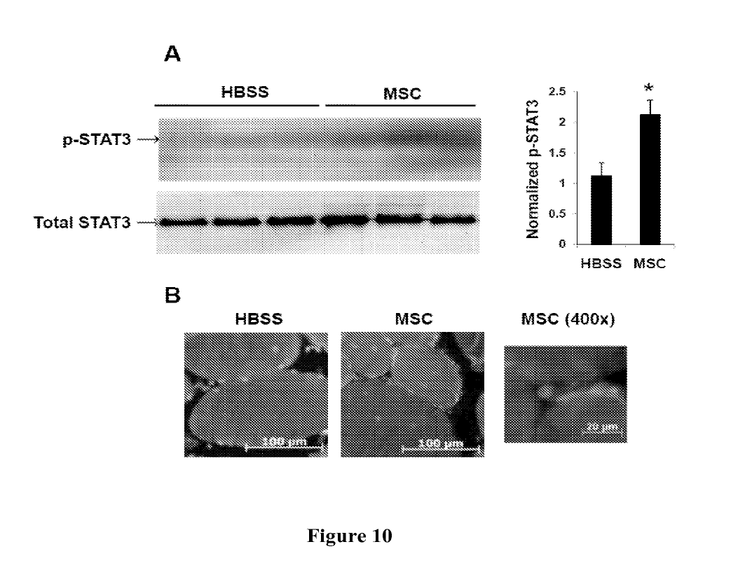

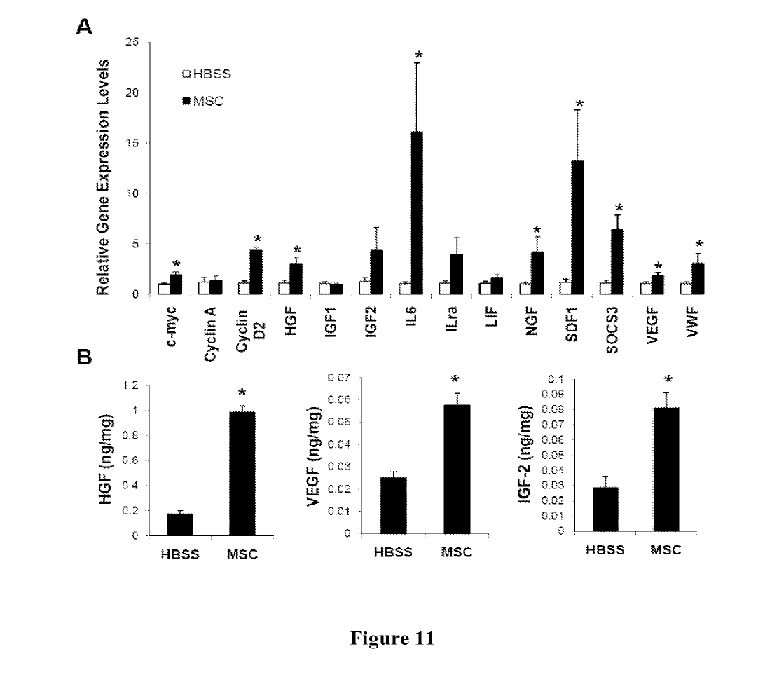

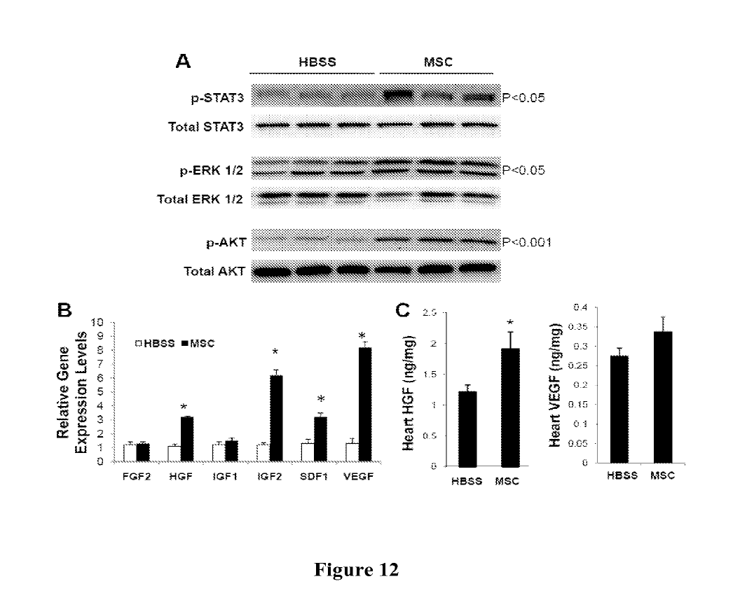

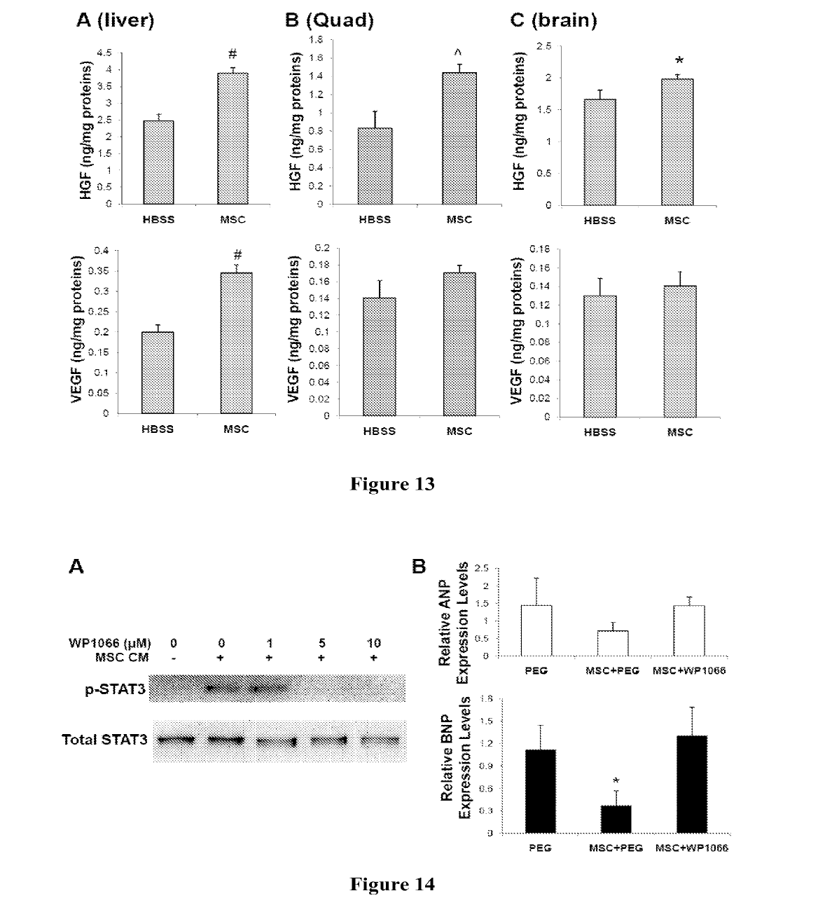

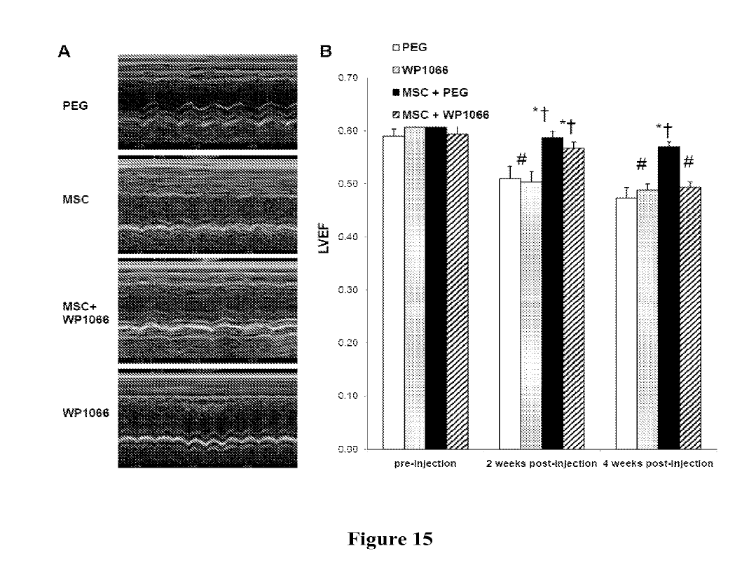

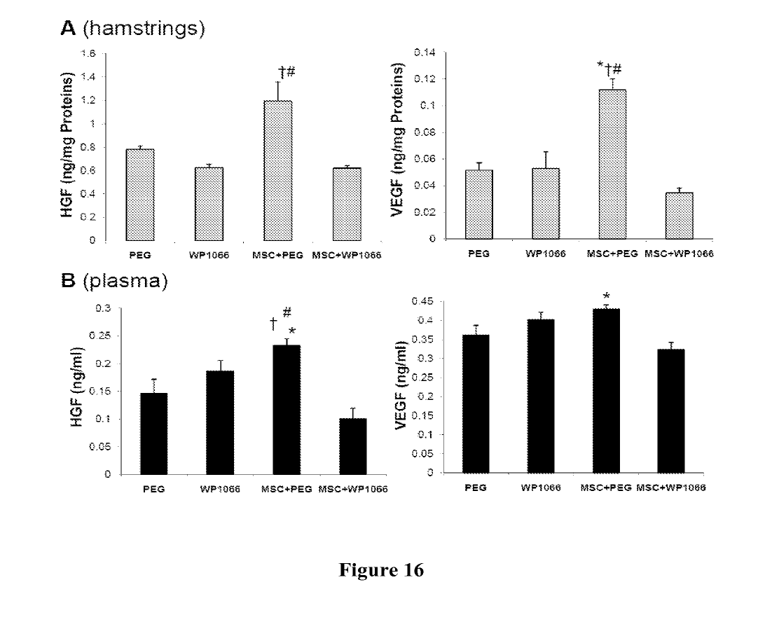

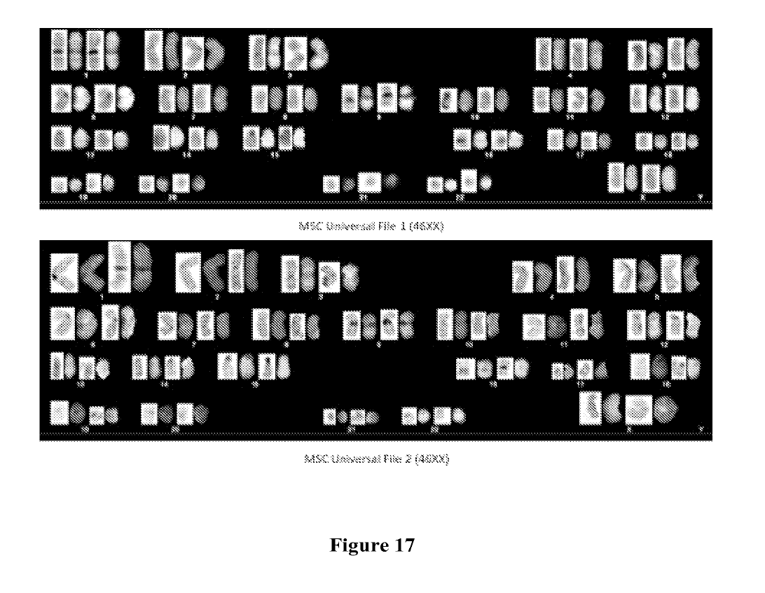

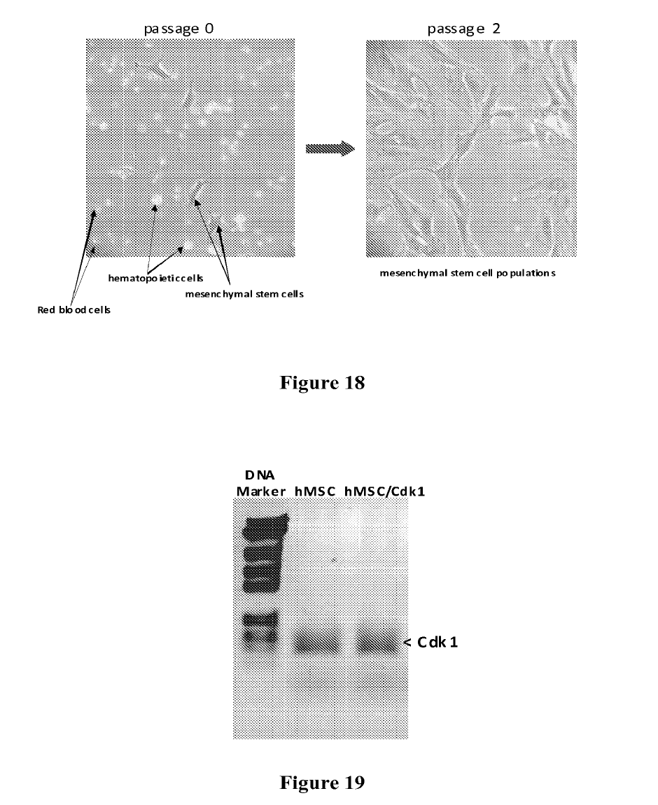

We further tested the cardiac therapeutic effect of the conditioned medium using the injection protocol and the hamster heart failure model described previously. We compared the therapeutic effects mediated by the cell and the medium. Both the cell and medium caused a similar increase in cardiomyocyte nuclear density (FIG. 2) and capillary density (FIG. 3) in the failing hamster heart, indicating active cardiac regeneration after therapy. We thus conclude that the conditioned medium derived from the engineered MSC line is therapeutically active.

While the foregoing description of the invention has been shown and described with reference to certain preferred embodiments thereof, it will be understood by those skilled in the art that various changes in form and details may be made therein without departing from the spirit and scope of the invention as described.

1. A mammalian mesenchymal stem cell in culture into which has been introduced a polynucleotide encoding for a mammalian Cdk1 protein.

2. The cell of claim 1, wherein the cell is non-tumorigenic and can be maintained stably in culture for at least 20 passages.

3. The cell of claim 1 wherein the mesenchymal stem cell is human cell.

4. The cell of claim 3, wherein the cell is obtained from human bone marrow.

5. The cell of claim 1, wherein the cell has not been passaged in culture before introduction of the polynucleotide.

6. The cell of claim 1, wherein the cell has been passaged once in culture before introduction of the polynucleotide.

7. The cell of claim 1, wherein the cell is cultured in a medium free of animal serum.

8. A conditioned medium which is obtained by incubating a plurality of mesenchymal stem cells from claim 1 in a culture medium which is free of non-human animal serum but contains human serum or plasma.

9. The conditioned medium of claim 8 which is free of cells or cellular debris.

10. The conditioned medium of claim 9 further comprising a pharmaceutical carrier.

11. The conditioned medium of claim 8, wherein the cells are incubated for at least 3 hours.

12. A method for tissue regeneration comprising the step of administering to an individual in need of treatment, a composition comprising the conditioned medium of claim 8 or a fraction thereof.

13. The method of claim 12, wherein the individual is in need of cardiac tissue repair.

14. The method of claim 12, wherein the composition comprises conditioned medium which has been obtained by incubating the mesenchymal stem cells with a culture medium comprising plasma obtained from the individual.
